(19) United States
(12) Patent Application Publication
(10) Pub. No.: US 2005/0096273 A1
(43) Pub. Date: May 5, 2005

(54) Regulation of matrix metalloproteinases by PSP94 family members

(76) Inventors: Chandra J. Panchal, London (CA); Luc Daigneault, Laval (CA); Robert Hawkins, Prestbury (GB); Marcia Ruiz, Ste-Genevieve (CA); Seema Garde, Montreal (CA)

Correspondence Address: Patent Administrator;Testa, Hurwitz & Thibeault, LLP, High Street Tower, 125 High Street, Boston, MA, 02110, US

(21) Appl. No.: 10/948,229

(22) Filed: Sep. 24, 2004

(30) Foreign Application Priority Data
Sep. 26, 2003 (CA) 2,441,695

(51) Int. Cl. A61K 38/54; C12N 9/99

(52) U.S. Cl. 514/12; 435/184

(57) ABSTRACT

Matrix metalloproteinases (MMPs) play an important role in morphogenesis, angiogenesis, wound healing, and in certain disorders such as rheumatoid arthritis, tumor invasion and metastasis. MMPs are thought to be regulated by a variety of cytokines, growth factors, hormones and phorbol esters. This regulation occurs on three levels; alteration of gene expression, activation of the latent zymogen and inhibition by the tissue inhibitors of metalloproteinases (TIMP). We report here a new agent that regulates the level of MMPs in patient. More particularly, members of the PSP94 family, when administered to patients having metastatic hormone resistant prostate cancer, promote a significant decrease in MMP plasma levels. The invention therefore relates to the use of a PSP94 family member for the treatment of a condition related to the activity or expression of MMPs.

### FIELD OF THE INVENTION

This invention relates to the regulation of matrix metalloproteinases (MMPs) by PSP94 family members. More particularly, the present invention relates to regulation of matrix metalloproteinases (MMPs) expression levels and/or activity by PSP94 family members. Also, the invention relates to the use of a PSP94 family member for the treatment of a condition related to the activity or expression of MMPs or pro-MMPs.

### BACKGROUND OF THE INVENTION

Matrix metalloproteinases (MMPs) play an important role in morphogenesis, angiogenesis, wound healing, and in certain disorders such as rheumatoid arthritis, tumor invasion and metastasis (Birkedal-Hansen, 1995, Curr. Opin. Cell Biol. 7:728-735). MMPs are involved, for example, in physiological function where rearrangements of basement membranes occur.

Five subfamilies of MMPs have been recognized: collagenases, gelatinases, stromelysins, matrilysins, and membrane-type MMPs (MT-MMPs). Most of these enzymes contain propeptide, catalytic and hemopexin domains and are involved in the degradation of collagens, proteoglycans and various glycoproteins. MMPs are secreted as inactive zymogens (pro-MMPs) and their activation seems to be a prerequisite for their function. In vivo activation of pro-MMPs involves the removal of the propeptide by serine proteases (e.g., trypsin, plasmin, etc.). Stimulation or repression of most pro-MMP synthesis is regulated at the transcriptional level by growth factors and cytokines.

Post-translational regulation of MMP activity, on the other hand, is controlled by tissue inhibitors of MMPs (“TIMPs”), four of which have been characterized and designated as TIMP-1, TIMP-2, TIMP-3, and TIMP-4 (Gomez et al., 1997, Eur. J. Cell. Biol. 74:111-122). MMP-2 (gelatinase A) and MMP-9 (gelatinase B) hydrolyze basement membrane (extracellular matrix (ECM) protein and non-ECM protein (including collagen)) and have therefore been incriminated in the mechanism of tumor invasion and metastasis. MMP-9 is also involved in inflammation, atheroscelerotic plaque rupture, tissue remodeling, wound healing, mobilization of matrix-bound growth factors, processing of cytokines, pulmonary fibrosis and osteoarthritis (Fujisawa et al., J. Biochem. 125:966, 1999). Its expression correlates, for example, with the desmoplasia (abnormal collagen deposition) that accompanies pancreatic cancer, with the metastasis to lymph nodes by human breast carcinoma cells and with the invasion of regional vessels in giant cell tumors of bones. MMP-9 may be elevated in gingival crevicular fluid and saliva in patients with gingivitis and periodontal diseases. MMP-2 binds specifically to TIMP-2 while MMP-9 binds to TIMP-1.

Evidences show that MMPs are overexpressed in cancer cells. However, in situ hybridization results indicated that stromal fibroblasts found at the proximity of cancer cells as well as vascular cells, inflammatory cells such as macrophages and neutrophils and not only the cancer cells expresses some MMP family members. Thus, there is a significant role of other cells expressing MMP in the contribution to cancer progression.

Determination of MMP-9 activity and/or level is useful, for example, in the follow-up and in the assessment of prognosis in breast and lung cancer patients (Ranunculo, Int. J. Cancer; Iizasa, Clinical Cancer Research) suggesting a good correlation between MMP-9 with the tumor burden and the clinical status.

Prostate secretory protein (PSP94) constitutes one of the three predominant proteins found in human seminal fluid along with prostate specific antigen (PSA) and prostatic acid phosphatase (PAP). PSP94 has a molecular weight of 10.7 kDa and contains 10 cysteine residues. The cDNA and the gene coding for PSP94 have been cloned and characterized.

PSP94 inhibits tumor growth (see U.S. Pat. No. 5,428,011 to Seth et al., the entire content of which is incorporated herein by reference). Tumor inhibition by PSP94 fragment such as PCK3145, has also been observed in animal models (see International application No. PCT/CA01/01463 to Garde, S. et al., published under No.: WO02/33090, the entire content of which is incorporated herein by reference). PSP94 also reduces the development of skeletal metastasis (see International application No.: PCT/CA02/01737 to Rabbani, S. et al., published under No.: WO03/039576, the entire content of which is incorporated herein by reference). This latter characteristic was observed by a reduction in calcium levels following administration of PSP94 to animal modeling prostate cancer.

Follicle stimulating hormone seems to be involved in the regulation of some MMPs and TIMPs, at least in Sertoli cells (see for example; Mol. Cell. Endocrinol. 118:3746, 1996; Biol. Reprod. 62:1040-1046, 2000; Mol. Cell. Endocrinol. 189: 25-35, 2002). In testis, follicle stimulating hormone (FSH) has been shown to induce the expression and secretion of MMP-2, MMP-9, TIMP-1 and TIMP-2 from Sertoli cells in vitro. In addition to its role in normal testicular and ovarian functions, FSH is also involved in stimulation of ovarian, endometrial and prostate tumor cell proliferation and is therefore implicated in tumor progression. PSP94 has been shown to inhibit FSH levels (Thakur et al., 1981, Ind. J. Exp. Biol. Vol. 19:303-313) and also interfere in the binding of FSH to its receptor using testicular membrane preparations (Vijayalakshmi et al., Int. J. Androl. (1981) 691-702).

### SUMMARY OF THE INVENTION

This invention relates to the regulation (either directly or indirectly) of matrix metalloproteinases (MMPs), (e.g., MMP-9) by PSP94 family members. More particularly, the present invention relates to the use of a PSP94 family member for the treatment of a condition related to the activity or expression of MMPs or pro-MMPs.

It was determined, in the present invention, that PSP94 family members reduce the level of MMPs in vivo. Results of MMP levels following administration of a PSP94 family member to patients having metastatic hormone resistant prostate cancer will thus be presented and discussed herein. This particularity of PSP94 family members was pointed out during a Phase IIa clinical trial designed to determine the safety and tolerability of PCK3145. Neither the safety/tolerability results nor survival status or disease status of patients will be discussed herein. However, for the purpose of clarity the trial design will be presented herein.

In one aspect, the present invention relates to a compound member of the PSP94 family for use in the treatment of a condition related to the activity or the expression of a protease (e.g., a serine protease). The condition may happen through the activity or expression of the protease itself or onto another factor (e.g., a factor which may be part of a cascade of event activated by the protease) which may be responsible for the condition.

In another aspect, the present invention provides a compound member of the PSP94 family for use in the treatment of a condition related, for example, to the activity or to the expression of a polypeptide which may be, for example, selected from the group consisting of matrix metalloproteinases and pro-matrix metalloproteinases.

It is to be understood herein that a member of the PSP94 family may be selected, for example, from the group consisting of PSP94 (SEQ ID NO.:1), a PSP94 fragment, a PSP94 derivative, a PSP94 analogue, PCK3145 (SEQ ID NO.:5), a PCK3145 fragment, a PCK3145 derivative and a PCK3145 analogue. A PCK3145 derivative may be, for example, as defined in SEQ ID NO.:7. PSP94 family members therefore also include, for example, SEQ ID NO.:2, SEQ ID NO.: 3, SEQ ID NO.:4, SEQ ID NO.:6, as well as SEQ ID NO.: 9 to 91.

More particularly, the member of the PSP94 family (PSP94 family member) may be selected, for example, from the group consisting of;
a) SEQ ID NO.:1,
b) a SEQ ID NO.: 1 derivative which may be able to reduce (in a tissue, a cell or cell environment (e.g., extracellular environment)) the activity or the level of expression of a polypeptide selected from the group consisting of a pro-matrix metalloproteinase and a matrix metalloproteinase,
c) a SEQ ID NO.:1 fragment which may be able to reduce the activity or the level of expression of a polypeptide selected from the group consisting of a pro-matrix metalloproteinase and a matrix metalloproteinase,
d) SEQ ID NO.:1 analogue which may be able to reduce the activity or the level of expression of a polypeptide selected from the group consisting of a pro-matrix metalloproteinase and a matrix metalloproteinase,
e) SEQ ID NO.:5,
f) a SEQ ID NO.:5 derivative which may be able to reduce the activity or the level of expression of a polypeptide selected from the group consisting of a pro-matrix metalloproteinase and a matrix metalloproteinase,
g) a SEQ ID NO.:5 fragment which may be able to reduce the activity or the level of expression of a polypeptide selected from the group consisting of a pro-matrix metalloproteinase and a matrix metalloproteinase,
h) a SEQ ID NO.:5 analogue which may be able to reduce the activity or the level of expression of a polypeptide selected from the group consisting of a pro-matrix metalloproteinase and a matrix metalloproteinase,
i) SEQ ID NO.:7, and
j) combination of any one of a) through i) thereof.

In accordance with the present invention the SEQ ID NO.:1 fragment may be selected, for example, from the group consisting of SEQ ID NO.:4 and SEQ ID NO.:6.

Also in accordance with the present invention the SEQ ID NO.:1 derivative may be selected, for example, from the group consisting of SEQ ID NO.:2 and SEQ ID NO.:3.

In a further aspect, the present invention provides the use of a PSP94 family member for the treatment of a condition related to the expression or related to the (e.g., biological, enzymatic) activity of a polypeptide which may be selected, for example, from the group consisting of matrix metalloproteinases and pro-matrix metalloproteinases.

In yet a further aspect, the present invention relates to the use of a PSP94 family member for the manufacture of a medicament (or pharmaceutical composition) for the treatment of a condition related to the expression or related to the activity of a polypeptide which may be, for example, selected from the group consisting of matrix metalloproteinases and pro-matrix metalloproteinases.

In accordance with the present invention, the condition may be selected from the group consisting of angiogenesis, inflammation, atheroscelerotic plaque rupture, skin disease, uncontrolled tissue remodeling and pulmonary fibrosis or any other condition or utility described herein.

In yet another aspect, the present invention relates to the use of a PSP94 family member for reducing or controlling the development or spreading of metastasis or metastatic cancer (i.e, cancer progression to other (secondary) sites or spreading of tumor cells to other sites) other than skeletal metastasis.

In another aspect, the present invention relates to the use of a PSP94 family member for the promotion of wound healing, for reducing (inhibiting) angiogenesis, for reducing (preventing) inflammation, for preventing atheroscelerotic plaque rupture, for skin treatment, for treating osteoarthritis, for treating pulmonary fibrosis or for the inhibition of (unwanted) hair growth.

In a further aspect, the present invention provides a pharmaceutical composition for treating a condition which may be related to the activity and/or to the expression of a polypeptide, which may be, selected from the group consisting of matrix metalloproteinases and pro-matrix metalloproteinases, the pharmaceutical composition may comprise;
a PSP94 family member, and;
a pharmaceutically acceptable carrier.

In yet a further aspect, the present invention provides a method for treating a patient having a condition related to the activity and/or expression of a polypeptide selected from the group consisting of matrix metalloproteinases and pro-matrix metalloproteinases, the method comprising administering to the patient a compound which is a member of the PSP94 family.

In another aspect, the present invention relates to a method of treating a patient having a metastatic cancer or a metastasis other than skeletal metastasis, the method comprising administering to the patient a PSP94 family member.

In an additional aspect, the present invention relates to a matrix metalloproteinase regulation drug and/or a pro-matrix metalloproteinase regulation drug comprising a PSP94 family member.

In another aspect, the present invention provides a compound able to reduce the expression or activity of a polypeptide selected from the group consisting of a matrix metalloproteinases and a pro-matrix metalloproteinases, the compound may comprise or consist essentially of the amino acid sequence identified in SEQ ID NO.:5 and may further comprise a stabilizing group (e.g. a group increasing in vivo stability of the compound or polypeptide without affecting deleteriously the biological activity of the compound or polypeptide) covalently attached to an amino acid of the (SEQ ID NO.: 5) sequence.

In accordance with the present invention the group may be, for example, an acetylaminomethyl group attached to a sulfur atom of a cysteine or a polyethylene glycol (PEG) group attached to at least one amino acid of the sequence or any other modification which improves a desired property (e.g., stability) of the compoun/polypeptide.

In yet another aspect, the present invention provides a method for evaluating the efficacy of a treatment with a PSP94 family member in a patient having a metastatic cancer or metastasis, the method may comprise, for example, the steps of
a) collecting a serum sample from the patient after treatment of the patient with a PSP94 family member;
b) measuring the (serum) levels of a polypeptide which may be selected from the group consisting of MMP-9 and pro-MMP-9 in the sample obtained in step a) and;
c) comparing measured levels of step b) with another MMP-9 and/or pro-MMP-9 level selected from the group consisting of levels measured from a normal individual, standard levels or levels measured before treatment of the individual.

The method may also comprise the step of establishing the clinical outcome of the patient based on the comparison of the measured levels.

In accordance with the present invention, the method for evaluating the efficacy of a PSP94 treatment may also measure any other parameters which might correlate with the level of expression of the polypeptide (MMP-9 and/or pro-MMP-9) such as for example, RNA levels.

In accordance with the present invention, the matrix metalloproteinase may be, for example, MMP-2 or may be MMP-9 or any other MMPs. Also in accordance with the present invention, the pro-matrix metalloproteinase may be, for example, pro-MMP-2 or may be pro-MMP-9 or any other pro-MMPs.

A “PSP94 family member” or “a member of the PSP94 family” is to be understood herein as any polypeptide originating from PSP94. For example, “PSP94 family members” may comprise wild type PSP94 (SEQ ID NO.:1) a PSP94 fragment, a PSP94 derivative, a PSP94 analogue, PCK3145 (SEQ ID NO.:5), a PCK3145 fragment, a PCK3145 derivative, a PCK3145 analogue, etc.

A “fragment” is to be understood herein as a polypeptide originating from a portion of an original or parent sequence. Fragments encompass polypeptides having truncations of one or more amino acids, wherein the truncation may originate from the amino terminus (N-terminus), carboxy terminus (C-terminus), or from the interior of the protein. A fragment may comprise the same sequence as the corresponding portion of the original sequence. For example, SEQ ID NO.: 4, SEQ ID NO.: 5 and SEQ ID NO.: 6 fall into the definition of “a PSP94 fragment”; when considering PSP94 (SEQ ID NO.:1) as an original sequence.

A “derivative” is to be understood herein as a polypeptide originating from an original sequence or from a portion of an original sequence and which may comprise one or more modification; for example, one or more modification in the amino acid sequence (e.g., an amino acid addition, deletion, insertion, substitution etc.), one or more modification in the backbone or side-chain of one or more amino acid, or an addition of a group or another molecule to one or more amino acids (side-chains or backbone). For example, SEQ ID NO.: 2, SEQ ID NO.: 3 and SEQ ID NO.: 7 fall into the definition of “a PSP94 derivative”; when considering PSP94 (SEQ ID NO.:1) as an original sequence.

It is to be understood herein that SEQ ID NO.: 7 may fall into the definition of “a PCK3145 derivative” or “SEQ ID NO.:5 derivative) when considering PCK3145 (SEQ ID NO.:5) as an original sequence. The addition of polyethylene glycol group (i.e., pegylation) to PCK3145 (SEQ ID NO.:5 or SEQ ID NO.: 7) also falls within the definition of “a PCK3145 derivative”.

An “analogue” is to be understood herein as a molecule having a biological activity and chemical structure similar to that of a polypeptide described herein. An “analogue” may have sequence similarity with that of an original sequence or a portion of an original sequence and may also have a modification of its structure as discussed herein. For example, an “analogue” may have at least 90% sequence similarity with an original sequence or a portion of an original sequence. An “analogue” may also have, for example; at least 70% or even 50% sequence similarity (or less, i.e., at least 40%) with an original sequence or a portion of an original sequence. Also, an “analogue” may have, for example, 50% sequence similarity to an original sequence with a combination of one or more modification in a backbone or side-chain of an amino acid, or an addition of a group or another molecule, etc.

Thus, biologically active polypeptides in the form of the original polypeptides, fragments (modified or not), analogues (modified or not), derivatives (modified or not), homologues, (modified or not) of PSP94 and PCK3145 are encompassed by the present invention.

Therefore, any polypeptide having a modification compared to an original polypeptide (e.g., PSP94, PCK3145) which does not destroy significantly a desired biological activity is encompassed herein. It is well known in the art, that a number of modifications may be made to the polypeptides of the present invention without deleteriously affecting their biological activity. These modifications may, on the other hand, may keep or increase the biological activity of the original polypeptide or may optimize one or more of the particularity (e.g. stability, bioavailability, etc.) of the polypeptides of the present invention which, in some instance might be desirable. Polypeptides of the present invention comprises for example, those containing amino acid sequences modified either by natural processes, such as posttranslational processing, or by chemical modification techniques which are known in the art. Modifications may occur anywhere in a polypeptide including the polypeptide backbone, the amino acid side-chains and the amino- or carboxy-terminus. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslational natural processes or may be made by synthetic methods. Modifications comprise for example, without limitation, pegylation, acetylation, acylation, addition of acetomidomethyl (Acm) group, ADP-ribosylation, alkylation, amidation, biotinylation, carbamoylation, carboxyethylation, esterification, covalent attachment to fiavin, covalent attachment to a heme moiety, covalent attachment of a nucleotide or nucleotide derivative, covalent attachment of drug, covalent attachment of a marker (e.g., fluorescent, radioactive, etc.), covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination, etc. It is to be understood herein that more than one modification to the polypeptides described herein are encompassed by the present invention to the extent that the biological activity is similar to the original (parent) polypeptide.

As discussed above, polypeptide modification may comprise, for example, amino acid insertion (i.e., addition), deletion and substitution (i.e., replacement), either conservative or non-conservative (e.g., D-amino acids, desamino acids) in the polypeptide sequence where such changes do not substantially alter the overall biological activity of the polypeptide which is to reduce the level of expression of matrix metalloproteinases or pro-matrix metalloproteinases and/or to reduce their enzymatic activity.

Example of substitutions may be those, which are conservative (i.e., wherein a residue is replaced by another of the same general type or group) or when wanted, non-conservative (i.e., wherein a residue is replaced by an amino acid of another type). In addition, a non-naturally occurring amino acid may substitute for a naturally occurring amino acid (i.e., non-naturally occurring conservative amino acid substitution or a non-naturally occurring non-conservative amino acid substitution).

As is understood, naturally occurring amino acids may be sub-classified as acidic, basic, neutral and polar, or neutral and non-polar. Furthermore, three of the encoded amino acids are aromatic. It may be of use that encoded polypeptides differing from the determined polypeptide of the present invention contain substituted codons for amino acids, which are from the same type or group as that of the amino acid be replaced. Thus, in some cases, the basic amino acids Lys, Arg and His may be interchangeable; the acidic amino acids Asp and Glu may be interchangeable; the neutral polar amino acids Ser, Thr, Cys, Gln, and Asn may be interchangeable; the non-polar aliphatic amino acids Gly, Ala, Val, Ile, and Leu are interchangeable but because of size Gly and Ala are more closely related and Val, Ile and Leu are more closely related to each other, and the aromatic amino acids Phe, Trp and Tyr may be interchangeable.

It should be further noted that if the polypeptides are made synthetically, substitutions by amino acids, which are not naturally encoded by DNA (non-naturally occurring or unnatural amino acid) may also be made.

A non-naturally occurring amino acid is to be understood herein as an amino acid which is not naturally produced or found in a mammal. A non-naturally occurring amino acid comprises an amino acid having an acetylaminomethyl group attached to a sulfur atom of a cysteine, a pegylated amino acid, etc. The inclusion of a non-naturally occurring amino acid in a defined polypeptide sequence will therefore generate a derivative of the original polypeptide. Non-naturally occurring amino acids (residues) include also the omega amino acids of the formula NH₂(CH₂)ₙCOOH wherein n is 2-6. Neutral nonpolar amino acids, such as sarcosine, t-butyl alanine, t-butyl glycine, N-methyl isoleucine, norleucine, etc. Phenylglycine may substitute for Trp, Tyr or Phe; citrulline and methionine sulfoxide are neutral nonpolar, cysteic acid is acidic, and omithine is basic. Proline may be substituted with hydroxyproline and retain the conformation conferring properties.

It is known in the art that analogues may be generated by substitutional mutagenesis and retain the biological activity of the polypeptides of the present invention. These analogues have at least one amino acid residue in the protein molecule removed and a different residue inserted in its place. For example, one site of interest for substitutional mutagenesis may include but are not restricted to sites identified as the active site(s), or immunological site(s). Other sites of interest may be those, for example, in which particular residues obtained from various species are identical. These positions may be important for biological activity. Examples of substitutions identified as “conservative substitutions” are shown in table 1. If such substitutions result in a change not desired, then other type of substitutions, denominated “exemplary substitutions” in table 1, or as further described herein in reference to amino acid classes, are introduced and the products screened.

In some cases it may be of interest to modify the biological activity of a polypeptide by amino acid substitution, insertion, or deletion. For example, modification of a polypeptide may result in an increase in the polypeptide's biological activity, may modulate its toxicity, may result in changes in bioavailability or in stability, or may modulate its immunological activity or immunological identity. Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:
(1) hydrophobic: norleucine, methionine (Met), Alanine (Ala), Valine (Val), Leucine (Leu), Isoleucine (Ile)
(2) neutral hydrophilic: Cysteine (Cys), Serine (Ser), Threonine (Thr)
(3) acidic: Aspartic acid (Asp), Glutamic acid (Glu)
(4) basic: Asparagine (Asn), Glutamine (Gln), Histidine (His), Lysine (Lys), Arginine (Arg)
(5) residues that influence chain orientation: Glycine (Gly), Proline (Pro); and aromatic: Tryptophan (Trp), Tyrosine (Tyr), Phenylalanine (Phe).

Non-conservative substitutions will entail exchanging a member of one of these classes for another.

TABLE 1
Preferred amino acid substitution

| Original residue | Exemplary substitution | Conservative substitution |
|---|---|---|
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, | Leu |
| | norleucine | |

Example of biologically active analogues of PCK3145 (SEQ ID NO: 5) exemplified by amino acid substitutions is illustrated below.

(SEQ ID NO.:88)

| | |
|---|---|
| Position | 1 5 10 15 |
| PCK3145 | E W Q T D N C E T C T C Y E T |
| | X₁ W Q X₂ D X₁ C X₁ X₂ C X₂ C X₃ X₁ X₂ |

For example, X₁ may be glutamic acid (i.e., glutamate) (Glu), aspartic acid (aspartate) (Asp), or asparagine (Asn), X₂ may be threonine (Thr) or serine (Ser) and X₃ may be tyrosine (Tyr) or phenylalanine (Phe). Any replacement of an original residue in SEQ ID NO.:5 with a conserved amino acid (i.e. conservative substitution) is encompassed by the present invention.

Polypeptides may be either naturally occurring (that is to say, substantially purified or isolated from a natural source) or synthetic (for example, by performing site-directed mutagenesis on the encoding DNA or made by other synthetic methods such as chemical synthesis). It is thus apparent that the polypeptides of the invention can be either naturally occurring or recombinant (that is to say prepared from the recombinant DNA techniques) or made by chemical synthesis (e.g., organic synthesis).

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

Further, as used herein “pharmaceutically acceptable carrier” or “pharmaceutical carrier” are known in the art and include, but are not limited to, 0.01-0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

It is to be understood herein that the biological activity of a desired polypeptide may be determined by contacting a cell expressing a metalloproteinase (e.g., MMP-9) and/or pro-metalloproteinase (e.g., pro-MMP-9) with a polypeptide of the present invention (a PSS94 family member (e.g.: original polypeptide, fragment, derivative, analogue, and/or any modified form of an original polypeptide, fragment, derivative or analogue) and, following incubation of the polypeptide and cell, evaluating the levels (inside the cell or in the extracellular environment (supernatant or blood (plasma or serum))) of expression of the metalloproteinase by western blot or the enzymatic activity of the metalloproteinase by zymography as described herein or by any other techniques known in the art to be representative of metalloproteinase activity or expression (e.g., northern blot, PCR, immunochemistry methods, etc.). A reduction of the level of expression or enzymatic activity of a metalloproteinase (and/or pro-metalloproteinase) will identify a biologically active polypeptide.

It is to be understood herein, that if a “range” or “group” of substances (e.g. amino acids), substituents” or the like is mentioned or if other types of a particular characteristic (e.g. temperature, pressure, chemical structure, time, etc.) is mentioned, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. Thus, for example,
with respect to a temperature greater than 100° C., this is to be understood as specifically incorporating herein each and every individual temperature state, as well as sub-range, above 100° C., such as for example 101° C., 105° C. and up, 110° C. and up, 115° C. and up, 110 to 135° C., 115° C. to 135° C., 102° C. to 150° C., up to 210° C., etc.;
   and similarly with respect to other parameters such as, concentrations, elements, etc. . . .

It is in particular to be understood herein that the polypeptides of the present invention each include each and every individual polypeptide described thereby as well as each and every possible mutant, variant, homolog, analogue or else whether such mutant, variant, homolog, analogue or else is defined as positively including particular polypeptides, as excluding particular polypeptides or a combination thereof; for example an exclusionary definition for a polypeptide analogue (e.g. X₁WQX₂DX₁CX₁X₂CX₂CX₃X₁X₂ (SEQ ID NO.88)) may read as follows: “provided that when one of X₁ is glutamic acid and X₂ is threonine X₃ may not be phenylalanine”. Another type of exclusionary definition may read “other than skeletal metastasis” or “provided that said condition is not skeletal metastasis”.

It is also to be understood herein that “g” or “gm” is a reference to the gram weight unit; that “C” is a reference to the Celsius temperature unit.

### BRIEF DESCRIPTION OF DRAWINGS

In drawings which illustrates exemplary embodiment of the present invention;

FIG. 1 is a picture of a zymography gel showing the effect of the PCK3145 derivative (SEQ ID NO.:7) on MMP-9 levels and activity on collagen type 1-treated MatLyLu cells (first lane:marker; second lane:cells; third lane:cells and collagen; fourth lane:cells, collagen and 500 μg/ml of SEQ ID NO.:7; fifth lane:cells, collagen and 1 mg/ml of SEQ ID NO.:7), and;

FIG. 2 is a picture of a western blot membrane showing the effect of the PCK3145 derivative (SEQ ID NO.:7) on MMP-9 expression level (fist lane: MMP9 standard; second lane:cells; third lane:cells and collagen; fourth lane:cells, collagen and 100 μg/ml of SEQ ID NO.:7; fifth lane: cells, collagen and 500 μg/ml of SEQ ID NO.:7; sixth lane: cells, collagen and 1 mg/ml of SEQ ID NO.:7).

### DETAILED DESCRIPTION OF THE INVENTION

Polypeptides which are members of the PSP94 family include; wild type PSP94 as defined in SEQ ID NO.: 1, a recombinant PSP94 as defined in SEQ ID NO.:2 and PSP94 derivatives, fragments and analogues as defined, for example in the amino acid SEQ ID NO.: 3, SEQ ID NO.:4, SEQ ID NO.:5, SEQ ID NO.:6 and SEQ ID NO.:7.

PCK3145 was chosen as a representative of the PSP94 family based on previous encouraging results of tumor growth inhibition observed in animals.

The wild type amino acid sequence of PCK3145 has been disclosed, for example, in international application No.: PCT/CA01/01463 and is defined herein in SEQ ID NO.: 5. A PCK3145 derivative has been generated by attaching an acetylaminomethyl group to the sulfur atom of each of the three cysteines of PCK3145. These groups stabilize the compound by preventing formation of peptide dimers or polymer by blocking the sulfhydryl group of the cysteines. This PCK3145 derivative is defined in SEQ ID NO.: 7. The drug was manufactured by Multiple Peptide Systems (3550) (General Atomics Court, San Diego, Calif.) using standard solid-phase peptide chemistry and lyophilized into a powder. Other type of synthesis or manufacture method may however be applied to make a peptide or polypeptide of the invention.

The reconstituted drug consists of a solution containing a target concentration of 20 mg/mL of PCK3145 derivative (SEQ ID NO.:5 derivative); SEQ ID NO.: 7, in a phosphate buffer at pH 7.4 for dilution in sterile saline (0.9% NaCl, BP) prior to intravenous administration. The solutions is filled into Type 1 glass vials, stoppered with Teflon®-faced butyl stoppers, and sealed with flip-off seals.

Clinical Trial

Trial Design

The clinical trial is a multiple ascending dose, open-label, Phase IIa study evaluating the safety and tolerability of PCK3145 derivative; SEQ ID NO.:7 administered intravenously in patients with metastatic hormone resistant prostatic cancer (HRPC). The study is not randomized. Patients have been enrolled sequentially and chronologically.

Inclusion Criteria

Patients had fulfilled the following criteria prior to receiving the first administration of the test drug:
Signed informed consent,
Have a histologically confirmed metastatic adenocarcinoma of the prostate,
Be characterized as a stage 1V prostatic cancer,
Have a metastatic hormone resistant prostatic cancer; resistance being defined as progressive disease after at least one hormonal therapy (orchiectomy, oestrogens, LHRH therapy). Progressive disease is defined in accordance with the recommendations of the Prostate Specific Antigen Working Group (Bubley, J. G., et al., J. of Clinic. Oncol. 17: 3461-3467, 1999) which defines progressive disease as:
   an increasing or development of new measurable disease or presence of new bone lesions on bone scan with a PSA level greater or equal to 5 ng/mL or
   two consecutive increases in PSA. The first increase should occur a minimum of 1 week from the reference value, and PSA level should be greater or equal to 5 ng/mL,
Be minimally symptomatic or asymptomatic defined as patients that may require chronic opioïd analgesics but have been on a stable pain management regimen for at least 4 weeks, Be males of at least 18 years of age,
Have baseline laboratory values as specified below:
   Aspartate aminiotransferase (ASAT) (S.I. Unit Value=Upper Normal Limit 42 u/L or <0.7 kat/L) less than or equal to 2.0 times the upper limit of normal and alanine aminotransferase (ALAT) (S.I. Unit Value=Upper Normal Limit <48 u/L or ≦0.8 μkat/L) less than or equal to 2.0 times the upper limit of normal Bilirubin less than 1.8 mg/dL (S. I. Unit Value=≦25.4 μmol./L) Creatinine less than 1.8 mg/dL (S.I. Unit Value=≦159 μmol./L) Platelets >100,000/mm³ (S.I. Unit Value=>100×10⁹ /L),
Have a life expectancy of at least 6 months,
Have a Karnofsky Performance status of 70% or greater,
Have the ability to understand the requirements of the study, provide written informed consent, abide by the study restrictions, and agree to return for the required assessments, Reliable contraception must be used throughout the study.
   Organisation of the Study

Four patients per cohort and 4 ascending doses were evaluated. The ascending doses were 5, 20, 40 and 80 mg/m². The dose escalation decision has been based on dose-limiting toxicity (DLT).

The 33-day cycle of treatment consisted of a PCK3145 derivative; SEQ ID NO.:7 administration three times per week (day 1, 3 and 5) for 26 days, followed by a 7 day post-treatment observation period. The maximum tolerated dose (MTD) is the dose level below the one inducing grade 3 or 4 drug related toxicity (DLT) in two patients from a cohort of a minimum of 4 patients. Only DLT's observed during the first cycle have been used for the dose escalation decision.

Each patient's participation consisted of the following study periods: a screening period held (between days −14 to −1), a baseline visit (at day 1) and before administration of the drug, a treatment period (from day 1 to day 26), a 7 days post-treatment observation period (from day 27 to day 33), a 6 month follow-up period where survival status, disease status and information about the occurrence of second primary tumors are assessed and a long term follow up period where survival status is assessed.

The treatment period consisted of intravenous administration of the PCK3145 derivative (SEQ ID NO.:5 derivative) i.e., SEQ ID NO.:7, three times per week (day 1, 3 and 5) for 26 consecutive days during which patients were closely monitored and undergone regular examination. After a week of treatment break and in the absence of toxicity and disease progression, patients optionally received additional treatment cycles.

Biological samples were drawn during different time points of the study for the purpose of safety monitoring and have been assayed for MMP-9 levels. Plasma samples were placed on dry ice and stored frozen (approximately −70° C.) and subsequently analyzed for total MMP-9 levels.

### EXAMPLE 1

In Vivo MMPs Measurements

MMP-9 Assay Methodology

An Elisa assay measuring total MMP-9 i.e., human active and pro-MMP-9, (Quantikine®, Cat. No.: DMP900, R&D Systems Inc.) was performed on plasma-heparin samples. Plasma samples have been collected from individuals at day 1 (before treatment) and at day 27 of each treatment cycle.

The Quantikine® MMP-9 immunoassay is a solid phase ELISA designed to measure total MMP-9 (92 kDa pro- and 82 kDa active forms) in serum, plasma, saliva, urine and cell culture supernatants. It is calibrated with CHO-cells expressed recombinant human pro-MMP-9 and the antibodies were raised against the recombinant factor. Both antibodies also recognize recombinant human active MMP-9. Natural human MMP-9 showed dose-response curves that were parallel to the standard curves obtained using the recombinant Quantikine® kit standards, indicating that the Quantikine® kit may be used to determine relative mass values of natural human MMP-9.

The assay employs the quantitative sandwich enzyme immunoassay technique. A monoclonal antibody specific for MMP-9 has been pre-coated onto a microplate. Standards and samples are added into the wells, and MMP-9 is thus bound by the immobilized antibody. After washing away unbound substances, an enzyme-linked polyclonal antibody specific for MMP-9 is added to the wells. Following a wash to remove unbound antibody-enzyme reagent, a substrate solution is added to the wells and color develops in proportion to the amount of total MMP-9 (pro and/or active) bound in the initial step. The color development is stopped and the intensity of the color is measured.

MMP-9 Assay Results

Results of MMP-9 levels in patient's plasma, before and after one or more treatment cycle with PCK3145 derivative; SEQ ID NO.: 7 are illustrated in Table 2.

Normal values of healthy volunteers were not determined in this study but Iizasa et al., has determined that the normal range of plasma MMP-9 concentrations is about 11.4 to 59.4 ng/ml. Based on theses values, patients were sub-divided into two categories; those having normal value of MMP-9 (below 100 μg/L) and those having an elevated level of MMP-9 (higher than 100 μg/L) at baseline (see column identified as DIC1 in Table2).

In the normal value MMP-9 category (patients identified as E, F, G, H and I), there was no significant decrease in MMP-9 levels after one cycle of treatment (column identified D27C1) compared to baseline levels. For patients E and G, no decrease in MMP-9 levels was observed compared to baseline values even after 2 cycles of treatment (column identified D27C2). There was still no MMP-9 decrease even after 3 cycles of treatment for patient E (D27C3).

In the elevated MMP-9 category (patients identified as A, B, C and D), a significant decrease was observed for each patient after only one cycle of treatment (see column identified as D27C1). For example a decrease of up to 89% in MMP-9 levels was observed for patient A compared to baseline levels. For patient B, the decrease in MMP-9 was 41% after cycle 1. For patients C and D the decrease at cycle 1 was 90% and 34% respectively.

This decrease was maintained for patients B and C who have received more treatment cycles (see columns identified as D27C2, D27C3 and D27C4). For example, at treatment cycle 2, patient B showed a reduction of 64% of its baseline level of MMP-9. A similar reduction was also measured for patient B at treatment cycle 3; i.e., a 65% reduction, and at treatment cycle 4; a 75% reduction. In the case of patient C, a reduction of 76% in MMP-9 levels was measured at cycle 2.

TABLE 2

| | | | | | | |
|---|---|---|---|---|---|---|
| | | | | | | Maximum |
| Patient | D1C1 | D27C1 | D27C2 | D27C3 | D27C4 | Reduction |

Elevated MMP-9: Baseline = 100 μg/L

| | | | | | | |
|---|---|---|---|---|---|---|
| A | 424 | 47.3 | N.A. | N.A. | N.A. | 89% |
| B | 156.5 | 91.6 | 55.6 | 54.5 | 39.4 | 75% |
| C | 155 | 14.9 | 37.7 | N.A. | N.A. | 90% |
| D | 130.2 | 85.2 | N.A. | N.A. | | 34% |

Normal MMP-9: Baseline = 100 μg/L

| | | | | | |
|---|---|---|---|---|---|
| E | 57.5 | 58 | 60.1 | 101.8 | N.A. |
| F | 53.2 | 73.1 | N.A. | N.A. | N.A. |
| G | 33.9 | 45.4 | 189.6 | | N.A. |
| H | 57.0 | 44.0 | 65 | | |
| I | 22.1 | 18.8 | | | |

N.A. = not applicable

### EXAMPLE 2

### In Vitro MMPs Measurements

In order to support in vivo results described in Example 1, zymography assays and western blot were performed on cell lines incubated with the PCK3145 derivative (SEQ ID NO.:7).

Zymography

Zymography is a technique generally used to analyze the activity of matrix metalloproteinases (MMPs) in biological samples. It involves the electrophoretic separation of proteins under denaturing (Sodium Dodecyl Sulfate (SDS)) but non-reducing conditions through a polyacrylamide gel containing gelatin (for example, 10% gel containing 1 mg/ml gelatin for MMP-9 and MMP-2 assays). The resolved proteins are re-natured by exchanging SDS with a non-ionic detergent such as Triton X-100 and the gel is incubated in an incubation buffer for activation of MMP-2 and MMP-9 (for example at 37° C. for 18 hrs). The gel is stained with Coomassie blue and the MMP-2 and MMP-9 bands may be visualized as clear bands against a blue background (i.e., the MMPs degrade the gelatin and are visualized as clear bands; pro MMP-2 is 68 kDa and pro-MMP-9 is 92 kDa). These bands can be quantified using densitometry.

Therefore, in the present experiment, 2.5×10⁵ MatLyLu tumor cells (American Type Culture Collection No.: JHU-5)) were seeded in T-25 flasks containing RPMI with 10% fetal bovine serum (FBS). After overnight incubation, the cells were washed once with serum free medium and treated with various concentrations of the PCK3145 derivative (500 ug/ml and 1 mg/ml) in the presence of 50 ug/ml collagen type-I in serum free RPMI for 72 hrs. Control cells received 50 ug/ml collagen or only serum free medium.

The media were collected after 72 hours of exposure to the PCK3145 derivative and subjected to gelatin zymography. Zymography for MMP-2 and MMP-9 was performed in SDS-polyacrylamide gel electrophoresis (SDS-PAGE) (10%) containing 0.1% gelatin (Invitrogen). Twenty-four microliters of culture media was mixed with non-reducing sample buffer and subjected to electrophoresis without boiling. After electrophoresis, gels were soaked for 30 minutes in 2.5% Triton X-100 solution with 2-3 washing steps. The gels were then incubated for 18 hours at 37° C. in buffer containing 50 mM Tris/HCl, pH 7.6, 50 mM NaCl, 10 mM CaCl₂ and 0.05% Brij-35. After incubation, the gels were stained with 0.2% Coomassie blue and de-stained until clear proteolytic bands appeared. Gels were scanned with Microtek flatbed scanner (Scanmaker 5 software; Microtek lab, Redondo Beach, Calif.). The band intensities were determined using the Image Quant software (version 5.0) from molecular Dynamics.

The MMP-9 and MMP-2 gelatinase zymography standard were purchased from Chemicon (catalogue no. CC073). One nanogram of purified human pro-MMP-2 and pro-MMP-9 standards were used in every gel run.

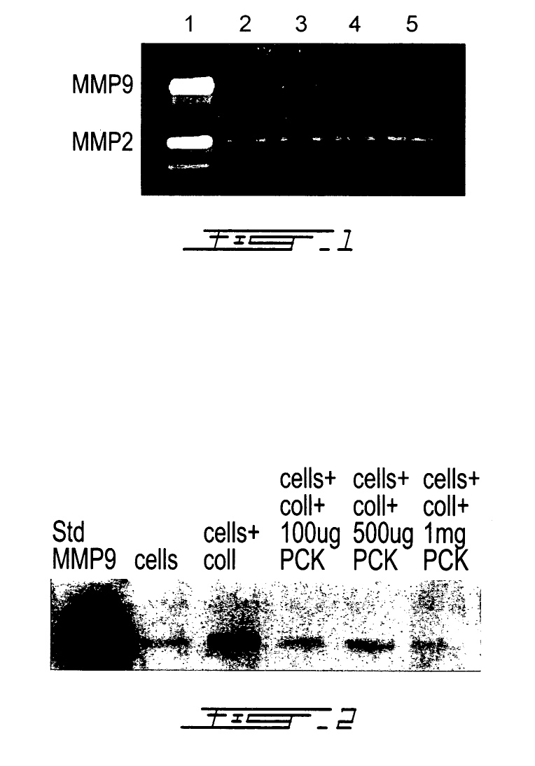

Results of this experiment are illustrated in FIG. 1 and indicate that PCK3145 treatment of MatLyLu cells resulted in a dose-dependent reduction of MMP-9 secreted to the cell culture media, as detected by zymography.

Western Blot

A western blot experimen was performed in a separate experiment in which MatLyLu cells were treated with 10 ug/ml, 500 ug/ml and 1 mg/ml of the PCK3145 derivative for 72 hrs. At the end of the experiment, the media were collected and concentrated 5 times using Amicon centrifugal filter devices (3500 molecular weight cut-off).

Twenty five microliters samples were separated on SDS-PAGE gel under reducing conditions using pre-cast gels of 4-12% Bis-Tris (Invitrogen). Following elcetrophoresis, the proteins were transferred on nitrocellulose membrane. Non-specific binding sites were blocked using 5% skimmed milk in 10 mM phosphate buffer saline (PBS) containing 0.05% Tween-20 for 1 hour at room temperature. The membrane was later incubated with a primary antibody (monoclonal, RDI-MMP-9abm-2A5) at a concentration of lug/ml (in 10 mM PBS, containing 0.5% bovine serum albumin (BSA) and 0.05% Tween-20) for 3 hours at room temperature.

The membranes were washed three times in PBS (5 minutes each wash) to remove non-specific binding and they were incubated with the secondary antibody (Rabbit anti-mouse IgG horseradish peroxidase-conjugated (Dako no. 0260)) at a dilution of 1:5000 for one hour. Detection of specific MMP-9 protein was made by incubating the membrane in ECL™ reagent (electro-chemoluminescence, Roche) and exposing to the X-ray film.

Results of this experiment are illustrated in FIG. 2 and again indicate that treatment of MatLyLu cells PCK3145 derivative resulted in a dose-dependent reduction of MMP-9 levels.

The effect of PSP94 family members on MMP-9 makes them useful for reduction of cancer spreading and invasion of any type of cancer and not only for reduction of skeletal metastasis as disclosed and claimed in International application No.: PCT/CA02/01737. For example, PSP94 family members are useful for the treatment of advanced metastatic diseases such as metastatic hormone resistant prostatic cancer.

In addition, PSP94 family members may be used not only for cancer related therapy but also for the treatment of any condition related to MMPs or pro-MMPs such as, for example, conditions related to the activity or expression (high concentration) of MMPs and/or pro-MMPs.

Inhibition of MMPs by PSP94, PSP94 derivatives, PCK3145, PCK3145 derivatives, analogues and homologues thereof may therefore find utility in cancer treatment, wound healing, anti-angiogesis, anti-inflammation, anti-osteoarthritis, inhibition of hair growth, reduction of degradation of some cytokine (e.g., IFN-beta) as well as for skin treatment (e.g., prevention of blistering photo-aging, psoriasis), wound healing, tissue remodeling, pulmonary fibrosis, etc.

Without trying to be exhaustive, it is to be understood herein, that inhibition of MMPs may be caused by one or more of the following;
an inhibition of MMP activity (e.g., binding of an inhibitor at the active site or any other site that will reduce its activity, affect its structure, stability, etc.),
an inhibition or reduction in MMP expression (inhibition of transcription of the gene, silencing of the gene, inhibition of translation of the mRNA, interference with the mRNA stability, transport, etc.),
an inhibition or reduction in pro-MMP activation,
a dominant negative mutant or an antagonist (e.g., which may compete for any molecules involved in the activity of MMPs (e.g., substrate, receptor, etc.),
a decrease in the MMP half-life, stability, etc.,
an increase in the expression, stability, of MMP's natural inhibitors; TIMPs or an increase in the stability, translation efficiency of TIMP's mRNA,
an increase in stability of the MMP-TIMP complex, etc.,
an effect on other factor(s) which influence any one of the above.

It is therefore understood herein that PSP94, PSP94 fragments, derivatives, analogues and homologues thereof may be involved in any one of the steps described above (through a direct mechanism or through an indirect mechanism). For example PCK3145 may have an anti-angiogenic effect by preventing VEGF production which is controlled by MMP-9.

The content of each publication, patent and patent application mentioned in the present application is incorporated herein by reference.

Although the present invention has been described in details herein and illustrated in the accompanying drawings, it is to be understood that the invention is not limited to the embodiments described herein and that various changes and modifications may be effected without departing from the scope or spirit of the present invention.

1. A compound which is a member of the PSP94 family for use in the treatment of a condition related to the activity or the expression of a protease.

2. A compound as defined in claim 1, wherein said protease is selected from the group consisting of matrix metalloproteinases and pro-matrix metalloproteinases.

3. A compound as defined in claim 2, wherein said protease is selected from the group consisting of MMP-9 and pro-MMP-9.

4. The compound as defined in claim 3, wherein said member of the PSP94 family is selected from the group consisting of;
a) SEQ ID NO.:1,
b) a SEQ ID NO.:1 derivative able to reduce the activity or the level of expression of a polypeptide selected from the group consisting of a pro-matrix metalloproteinase and a matrix metalloproteinase,
c) a SEQ ID NO.:1 fragment able to reduce the activity or the level of expression of a polypeptide selected from the group consisting of a pro-matrix metalloproteinase and a matrix metalloproteinase,
d) SEQ ID NO.:1 analogue able to reduce the activity or the level of expression of a polypeptide selected from the group consisting of a pro-matrix metalloproteinase and a matrix metalloproteinase,
e) SEQ ID NO.:5,
f) a SEQ ID NO.:5 derivative able to reduce the activity or the level of expression of a polypeptide selected from the group consisting of a pro-matrix metalloproteinase and a matrix metalloproteinase,
g) a SEQ ID NO.:5 fragment able to reduce the activity or the level of expression of a polypeptide selected from the group consisting of a pro-matrix metalloproteinase and a matrix metalloproteinase,
h) a SEQ ID NO.:5 analogue able to reduce the activity or the level of expression of a polypeptide selected from the group consisting of a pro-matrix metalloproteinase and a matrix metalloproteinase,
i) SEQ ID NO.:7, and
j) combination of any one of a) through i) thereof.

5. The compound of claim 4, wherein said compound possesses the amino acid sequence defined in SEQ ID NO.:5.

6. The compound of claim 4, wherein said compound possesses the amino acid sequence defined in SEQ ID NO.:7.

7. The compound of claim 4, wherein said SEQ ID NO.:1 fragment is selected from the group consisting of SEQ ID NO.:4 and SEQ ID NO.:6.

8. The compound of claim 4, wherein said SEQ ID NO.:1 derivative is selected from the group consisting of SEQ ID NO.:2 and SEQ ID NO.:3.

9. The use of a PSP94 family member in the manufacture of a medicament for the treatment of a condition related to the expression or activity of a polypeptide selected from the group consisting of matrix metalloproteinases and pro-matrix metalloproteinases.

10. The use as defined in claim 9, wherein said polypeptide is selected from the group consisting of MMP-9 and pro-MMP-9.

11. The use as defined in claim 10, wherein said PSP94 family member is selected from the group consisting of;
a) SEQ ID NO.:1,
b) a SEQ ID NO.:1 derivative able to reduce the activity or the level of expression of a polypeptide selected from the group consisting of a pro-matrix metalloproteinase and a matrix metalloproteinase,
c) a SEQ ID NO.:1 fragment able to reduce the activity or the level of expression of a polypeptide selected from the group consisting of a pro-matrix metalloproteinase and a matrix metalloproteinase,
d) SEQ ID NO.:1 analogue able to reduce the activity or the level of expression of a polypeptide selected from the group consisting of a pro-matrix metalloproteinase and a matrix metalloproteinase,
e) SEQ ID NO.:5,
f) a SEQ ID NO.:5 derivative able to reduce the activity or the level of expression of a polypeptide selected from the group consisting of a pro-matrix metalloproteinase and a matrix metalloproteinase,
g) a SEQ ID NO.:5 fragment able to reduce the activity or the level of expression of a polypeptide selected from the group consisting of a pro-matrix metalloproteinase and a matrix metalloproteinase,
h) a SEQ ID NO.:5 analogue able to reduce the activity or the level of expression of a polypeptide selected from the group consisting of a pro-matrix metalloproteinase and a matrix metalloproteinase,
i) SEQ ID NO.:7, and
j) combination of any one of a) through i) thereof.

12. The use as define in claim 11, wherein said PSP94 family member possesses the amino acid sequence defined in SEQ ID NO.:5.

13. The use as defined in claim 11, wherein said PSP94 family member possesses the amino acid sequence defined in SEQ ID NO.:7.

14. The use as defined in claim 11, wherein said SEQ ID NO.:1 fragment is selected from the group consisting of SEQ ID NO.:4 and SEQ ID NO.:6.

15. The use as defined in claim 11, wherein said SEQ ID NO.:1 derivative is selected from the group consisting of SEQ ID NO.:2 and SEQ ID NO.:3.

16. A method of treating a patient having a metastatic cancer or metastasis other than skeletal metastasis, the method comprising administering to said patient a PSP94 family member.

17. The method of claim 16, wherein said PSP94 family member is selected from the group consisting of;
a) SEQ ID NO.:1,
b) a SEQ ID NO.:1 derivative able to reduce the activity or the level of expression of a polypeptide selected from the group consisting of a pro-matrix metalloproteinase and a matrix metalloproteinase,
c) a SEQ ID NO.:1 fragment able to reduce the activity or the level of expression of a polypeptide selected from the group consisting of a pro-matrix metalloproteinase and a matrix metalloproteinase,
d) SEQ ID NO.:1 analogue able to reduce the activity or the level of expression of a polypeptide selected from the group consisting of a pro-matrix metalloproteinase and a matrix metalloproteinase,
e) SEQ ID NO.:5,
f) a SEQ ID NO.:5 derivative able to reduce the activity or the level of expression of a polypeptide selected from the group consisting of a pro-matrix metalloproteinase and a matrix metalloproteinase,
g) a SEQ ID NO.:5 fragment able to reduce the activity or the level of expression of a polypeptide selected from the group consisting of a pro-matrix metalloproteinase and a matrix metalloproteinase,
h) a SEQ ID NO.:5 analogue able to reduce the activity or the level of expression of a polypeptide selected from the group consisting of a pro-matrix metalloproteinase and a matrix metalloproteinase,
i) SEQ ID NO.:7, and
j) combination of any one of a) through i) thereof.

18. The method of claim 17, wherein said PSP94 family member possesses the amino acid sequence defined in SEQ ID NO.:5.

19. The method of claim 17, wherein said PSP94 family member possesses the amino acid sequence defined in SEQ ID NO.:7.

20. The method of claim 17, wherein said SEQ ID NO.:1 fragment is selected from the group consisting of SEQ ID NO.:4 and SEQ ID NO.:6.

21. The method of claim 17, wherein said SEQ ID NO.:1 derivative is selected from the group consisting of SEQ ID NO.:2 and SEQ ID NO.:3.

22. A pharmaceutical composition for treating a condition related to the activity or expression of a polypeptide selected from the group consisting of matrix metalloproteinases and pro-matrix metalloproteinases, said pharmaceutical composition comprising;
a) a PSP94 family member, and;
b) a pharmaceutically acceptable carrier.

23. The pharmaceutical composition of claim 22, wherein said polypeptide is selected from the group consisting of MMP-9 and pro-MMP-9.

24. The pharmaceutical composition as defined in claim 23, wherein said PSP94 family member is selected from the group consisting of;
a) SEQ ID NO.:1,
b) a SEQ ID NO.:1 derivative able to reduce the activity or the level of expression of a polypeptide selected from the group consisting of a pro-matrix metalloproteinase and a matrix metalloproteinase,
c) a SEQ ID NO.:1 fragment able to reduce the activity or the level of expression of a polypeptide selected from the group consisting of a pro-matrix metalloproteinase and a matrix metalloproteinase,
d) SEQ ID NO.:1 analogue able to reduce the activity or the level of expression of a polypeptide selected from the group consisting of a pro-matrix metalloproteinase and a matrix metalloproteinase,
e) SEQ ID NO.:5,
f) a SEQ ID NO.:5 derivative able to reduce the activity or the level of expression of a polypeptide selected from the group consisting of a pro-matrix metalloproteinase and a matrix metalloproteinase,
g) a SEQ ID NO.:5 fragment able to reduce the activity or the level of expression of a polypeptide selected from the group consisting of a pro-matrix metalloproteinase and a matrix metalloproteinase,
h) a SEQ ID NO.:5 analogue able to reduce the activity or the level of expression of a polypeptide selected from the group consisting of a pro-matrix metalloproteinase and a matrix metalloproteinase,
i) SEQ ID NO.:7, and
j) combination of any one of a) through i) thereof.

25. The pharmaceutical composition as defined in claim 24, wherein said PSP94 family member possesses the amino acid sequence defined in SEQ ID NO.:5.

26. The pharmaceutical composition as defined in claim 24, wherein said PSP94 family member possesses the amino acid sequence defined in SEQ ID NO.:7.

27. The pharmaceutical composition as defined in claim 24, wherein said SEQ ID NO.:1 fragment is selected from the group consisting of SEQ ID NO.:4 and SEQ ID NO.:6.

28. The pharmaceutical composition as defined in claim 24, wherein said SEQ ID NO.:1 derivative is selected from the group consisting of SEQ ID NO.:2 and SEQ ID NO.:3.

29. A method for treating a patient having a condition related to the activity or expression of a polypeptide selected from the group consisting of matrix metalloproteinases and pro-matrix metalloproteinases, said method comprising administering to the patient a compound which is a member of the PSP94 family.

30. The method of claim 29, wherein said polypeptide is selected from the group consisting of a MMP-9 and a pro-MMP-9.

31. The method as defined in claim 30, wherein said member of the PSP94 family is selected from the group consisting of;
a) SEQ ID NO.:1,
b) a SEQ ID NO.:1 derivative able to reduce the activity or the level of expression of a polypeptide selected from the group consisting of a pro-matrix metalloproteinase and a matrix metalloproteinase,
c) a SEQ ID NO.:1 fragment able to reduce the activity or the level of expression of a polypeptide selected from the group consisting of a pro-matrix metalloproteinase and a matrix metalloproteinase,
d) SEQ ID NO.:1 analogue able to reduce the activity or the level of expression of a polypeptide selected from the group consisting of a pro-matrix metalloproteinase and a matrix metalloproteinase,
e) SEQ ID NO.:5,
f) a SEQ ID NO.:5 derivative able to reduce the activity or the level of expression of a polypeptide selected from the group consisting of a pro-matrix metalloproteinase and a matrix metalloproteinase,
g) a SEQ ID NO.:5 fragment able to reduce the activity or the level of expression of a polypeptide selected from the group consisting of a pro-matrix metalloproteinase and a matrix metalloproteinase,
h) a SEQ ID NO.:5 analogue able to reduce the activity or the level of expression of a polypeptide selected from the group consisting of a pro-matrix metalloproteinase and a matrix metalloproteinase,
i) SEQ ID NO.:7, and
j) combination of any one of a) through i) thereof.

32. The method of claim 31, wherein said member of the PSP94 family possesses the amino acid sequence defined in SEQ ID NO.:5.

33. The method of claim 31, wherein said member of the PSP94 family possesses the amino acid sequence defined in SEQ ID NO.:7.

34. The method of claim 31, wherein said SEQ ID NO.:1 fragment is selected from the group consisting of SEQ ID NO.:4 and SEQ ID NO.:6.

35. The method of claim 31, wherein said SEQ ID NO.:1 derivative is selected from the group consisting of SEQ ID NO.:2 and SEQ ID NO.:3.

36. The method of claim 31, wherein said condition is selected from the group consisting of angiogenesis, inflammation, atheroscelerotic plaque rupture, skin disease, uncontrolled tissue remodeling and pulmonary fibrosis.

37. A compound able to reduce the expression or activity of a polypeptide selected from the group consisting of a matrix metalloproteinases and a pro-matrix metalloproteinases, said compound consisting essentially of the amino acid sequence identified in SEQ ID NO.:5 and further comprising a stabilizing group covalently attached to an amino acid of said sequence.

38. The compound as defined in claim 37, wherein said polypeptide is selected from the group consisting of a MMP-9 and a pro-MMP-9.

39. The compound as defined in claim 38, wherein said stabilizing group is an acetylaminomethyl group attached to a sulfur atom of a cysteine.

40. The compound as defined in claim 38, wherein said compound has the composition defined in SEQ ID NO.:7

41. A method for evaluating the efficacy of a treatment with a PSP94 family member in a patient having a metastatic cancer, the method comprising the steps of
a) collecting a serum sample from the patient after treatment of the patient with a PSP94 family member;
b) measuring the levels of a polypeptide selected from the group consisting of MMP-9 and pro-MMP-9 in the sample obtained in step a), and;
c) comparing measured levels of step b) with another MMP-9 and/or pro-MMP-9 level selected from the group consisting of levels measured from a normal individual, standard levels or levels measured before treatment of the individual.


SEQUENCE LISTING (91 sequences)

SEQ ID NO: 1 - protein, 94 aa, Homo sapiens
SCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 2 - protein, 102 aa, Artificial Sequence
  PSP94 derivative
EAEAYVEFSCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 3 - protein, 10 aa, Artificial Sequence
  PSP94 derivative
YTCSVSEPGI

SEQ ID NO: 4 - protein, 15 aa, Artificial Sequence
  PSP94 fragment
NEGVPGDSTRKCMDL

SEQ ID NO: 5 - protein, 15 aa, Artificial Sequence
  PCK3154
EWQTDNCETCTCYET

SEQ ID NO: 6 - protein, 19 aa, Artificial Sequence
  PSP94 fragment
IVVEKKDPKKTCSVSEWII

SEQ ID NO: 7 - protein, 15 aa, Artificial Sequence
  PSP94 derivative
EWQTDNCETCTCYET

SEQ ID NO: 8 - DNA, 285 bp, Artificial Sequence
  cDNA
tcatgctatttcatacctaatgagggagttccaggagattcaaccaggaaatgcatggatctcaaaggaaacaaacacccaataaactcggagtggcagactgacaactgtgagacatgcacttgctacgaaacagaaatttcatgttgcacccttgtttctacacctgtgggttatgacaaagacaactgccaaagaatcttcaagaaggaggactgcaagtatatcgtggtggagaagaaggacccaaaaaagacctgttctgtcagtgaatggataatctaa

SEQ ID NO: 9 - protein, 16 aa, Artificial Sequence
  PSP94 family member
EWQTDNCETCTCYETE

SEQ ID NO: 10 - protein, 17 aa, Artificial Sequence
  PSP94 family member
EWQTDNCETCTCYETEI

SEQ ID NO: 11 - protein, 18 aa, Artificial Sequence
  PSP94 family member
EWQTDNCETCTCYETEIS

SEQ ID NO: 12 - protein, 19 aa, Artificial Sequence
  PSP94 family member
EWQTDNCETCTCYETEISC

SEQ ID NO: 13 - protein, 20 aa, Artificial Sequence
  PSP94 family member
EWQTDNCETCTCYETEISCC

SEQ ID NO: 14 - protein, 21 aa, Artificial Sequence
  PSP94 family member
EWQTDNCETCTCYETEISCCT

SEQ ID NO: 15 - protein, 22 aa, Artificial Sequence
  PSP94 family member
EWQTDNCETCTCYETEISCCTL

SEQ ID NO: 16 - protein, 23 aa, Artificial Sequence
  PSP94 family member
EWQTDNCETCTCYETEISCCTLV

SEQ ID NO: 17 - protein, 24 aa, Artificial Sequence
  PSP94 family member
EWQTDNCETCTCYETEISCCTLVS

SEQ ID NO: 18 - protein, 25 aa, Artificial Sequence
  PSP94 family member
EWQTDNCETCTCYETEISCCTLVST

SEQ ID NO: 19 - protein, 26 aa, Artificial Sequence
  PSP94 family member
EWQTDNCETCTCYETEISCCTLVSTP

SEQ ID NO: 20 - protein, 27 aa, Artificial Sequence
  PSP94 family member
EWQTDNCETCTCYETEISCCTLVSTPV

SEQ ID NO: 21 - protein, 28 aa, Artificial Sequence
  PSP94 family member
EWQTDNCETCTCYETEISCCTLVSTPVG

SEQ ID NO: 22 - protein, 29 aa, Artificial Sequence
  PSP94 family member
EWQTDNCETCTCYETEISCCTLVSTPVGY

SEQ ID NO: 23 - protein, 30 aa, Artificial Sequence
  PSP94 family member
EWQTDNCETCTCYETEISCCTLVSTPVGYD

SEQ ID NO: 24 - protein, 31 aa, Artificial Sequence
  PSP94 family member
EWQTDNCETCTCYETEISCCTLVSTPVGYDK

SEQ ID NO: 25 - protein, 32 aa, Artificial Sequence
  PSP94 family member
EWQTDNCETCTCYETEISCCTLVSTPVGYDKD

SEQ ID NO: 26 - protein, 33 aa, Artificial Sequence
  PSP94 family member
EWQTDNCETCTCYETEISCCTLVSTPVGYDKDN

SEQ ID NO: 27 - protein, 34 aa, Artificial Sequence
  PSP94 family member
EWQTDNCETCTCYETEISCCTLVSTPVGYDKDNC

SEQ ID NO: 28 - protein, 35 aa, Artificial Sequence
  PSP94 family member
EWQTDNCETCTCYETEISCCTLVSTPVGYDKDNCQ

SEQ ID NO: 29 - protein, 36 aa, Artificial Sequence
  PSP94 family member
EWQTDNCETCTCYETEISCCTLVSTPVGYDKDNCQR

SEQ ID NO: 30 - protein, 37 aa, Artificial Sequence
  PSP94 family member
EWQTDNCETCTCYETEISCCTLVSTPVGYDKDNCQRI

SEQ ID NO: 31 - protein, 38 aa, Artificial Sequence
  PSP94 family member
EWQTDNCETCTCYETEISCCTLVSTPVGYDKDNCQRIF

SEQ ID NO: 32 - protein, 39 aa, Artificial Sequence
  PSP94 family member
EWQTDNCETCTCYETEISCCTLVSTPVGYDKDNCQRIFK

SEQ ID NO: 33 - protein, 40 aa, Artificial Sequence
  PSP94 family member
EWQTDNCETCTCYETEISCCTLVSTPVGYDKDNCQRIFKK

SEQ ID NO: 34 - protein, 41 aa, Artificial Sequence
  PSP94 family member
EWQTDNCETCTCYETEISCCTLVSTPVGYDKDNCQRIFKKE

SEQ ID NO: 35 - protein, 42 aa, Artificial Sequence
  PSP94 family member
EWQTDNCETCTCYETEISCCTLVSTPVGYDKDNCQRIFKKED

SEQ ID NO: 36 - protein, 43 aa, Artificial Sequence
  PSP94 family member
EWQTDNCETCTCYETEISCCTLVSTPVGYDKDNCQRIFKKEDC

SEQ ID NO: 37 - protein, 44 aa, Artificial Sequence
  PSP94 family member
EWQTDNCETCTCYETEISCCTLVSTPVGYDKDNCQRIFKKEDCK

SEQ ID NO: 38 - protein, 45 aa, Artificial Sequence
  PSP94 family member
EWQTDNCETCTCYETEISCCTLVSTPVGYDKDNCQRIFKKEDCKY

SEQ ID NO: 39 - protein, 46 aa, Artificial Sequence
  PSP94 family member
EWQTDNCETCTCYETEISCCTLVSTPVGYDKDNCQRIFKKEDCKYI

SEQ ID NO: 40 - protein, 47 aa, Artificial Sequence
  PSP94 family member
EWQTDNCETCTCYETEISCCTLVSTPVGYDKDNCQRIFKKEDCKYIV

SEQ ID NO: 41 - protein, 48 aa, Artificial Sequence
  PSP94 family member
EWQTDNCETCTCYETEISCCTLVSTPVGYDKDNCQRIFKKEDCKYIVV

SEQ ID NO: 42 - protein, 49 aa, Artificial Sequence
  PSP94 family member
EWQTDNCETCTCYETEISCCTLVSTPVGYDKDNCQRIFKKEDCKYIVVE

SEQ ID NO: 43 - protein, 50 aa, Artificial Sequence
  PSP94 family member
EWQTDNCETCTCYETEISCCTLVSTPVGYDKDNCQRIFKKEDCKYIVVEK

SEQ ID NO: 44 - protein, 51 aa, Artificial Sequence
  PSP94 family member
EWQTDNCETCTCYETEISCCTLVSTPVGYDKDNCQRIFKKEDCKYIVVEKK

SEQ ID NO: 45 - protein, 52 aa, Artificial Sequence
  PSP94 family member
EWQTDNCETCTCYETEISCCTLVSTPVGYDKDNCQRIFKKEDCKYIVVEKKD

SEQ ID NO: 46 - protein, 53 aa, Artificial Sequence
  PSP94 family member
EWQTDNCETCTCYETEISCCTLVSTPVGYDKDNCQRIFKKEDCKYIVVEKKDP

SEQ ID NO: 47 - protein, 54 aa, Artificial Sequence
  PSP94 family member
EWQTDNCETCTCYETEISCCTLVSTPVGYDKDNCQRIFKKEDCKYIVVEKKDPK

SEQ ID NO: 48 - protein, 55 aa, Artificial Sequence
  PSP94 family member
EWQTDNCETCTCYETEISCCTLVSTPVGYDKDNCQRIFKKEDCKYIVVEKKDPKK

SEQ ID NO: 49 - protein, 56 aa, Artificial Sequence
  PSP94 family member
EWQTDNCETCTCYETEISCCTLVSTPVGYDKDNCQRIFKKEDCKYIVVEKKDPKKT

SEQ ID NO: 50 - protein, 57 aa, Artificial Sequence
  PSP94 family member
EWQTDNCETCTCYETEISCCTLVSTPVGYDKDNCQRIFKKEDCKYIVVEKKDPKKTC

SEQ ID NO: 51 - protein, 58 aa, Artificial Sequence
  PSP94 family member
EWQTDNCETCTCYETEISCCTLVSTPVGYDKDNCQRIFKKEDCKYIVVEKKDPKKTCS

SEQ ID NO: 52 - protein, 59 aa, Artificial Sequence
  PSP94 family member
EWQTDNCETCTCYETEISCCTLVSTPVGYDKDNCQRIFKKEDCKYIVVEKKDPKKTCSV

SEQ ID NO: 53 - protein, 60 aa, Artificial Sequence
  PSP94 family member
EWQTDNCETCTCYETEISCCTLVSTPVGYDKDNCQRIFKKEDCKYIVVEKKDPKKTCSVS

SEQ ID NO: 54 - protein, 61 aa, Artificial Sequence
  PSP94 family member
EWQTDNCETCTCYETEISCCTLVSTPVGYDKDNCQRIFKKEDCKYIVVEKKDPKKTCSVSE

SEQ ID NO: 55 - protein, 62 aa, Artificial Sequence
  PSP94 family member
EWQTDNCETCTCYETEISCCTLVSTPVGYDKDNCQRIFKKEDCKYIVVEKKDPKKTCSVSEW

SEQ ID NO: 56 - protein, 63 aa, Artificial Sequence
  PSP94 family member
EWQTDNCETCTCYETEISCCTLVSTPVGYDKDNCQRIFKKEDCKYIVVEKKDPKKTCSVSEWI

SEQ ID NO: 57 - protein, 64 aa, Artificial Sequence
  PSP94 family member
EWQTDNCETCTCYETEISCCTLVSTPVGYDKDNCQRIFKKEDCKYIVVEKKDPKKTCSVSEWII

SEQ ID NO: 58 - protein, 16 aa, Artificial Sequence
  PSP94 family member
SEWQTDNCETCTCYET

SEQ ID NO: 59 - protein, 17 aa, Artificial Sequence
  PSP94 family member
NSEWQTDNCETCTCYET

SEQ ID NO: 60 - protein, 18 aa, Artificial Sequence
  PSP94 family member
INSEWQTDNCETCTCYET

SEQ ID NO: 61 - protein, 19 aa, Artificial Sequence
  PSP94 family member
PINSEWQTDNCETCTCYET

SEQ ID NO: 62 - protein, 20 aa, Artificial Sequence
  PSP94 family member
HPINSEWQTDNCETCTCYET

SEQ ID NO: 63 - protein, 21 aa, Artificial Sequence
  PSP94 family member
KHPINSEWQTDNCETCTCYET

SEQ ID NO: 64 - protein, 22 aa, Artificial Sequence
  PSP94 family member
NKHPINSEWQTDNCETCTCYET

SEQ ID NO: 65 - protein, 23 aa, Artificial Sequence
  PSP94 family member
GNKHPINSEWQTDNCETCTCYET

SEQ ID NO: 66 - protein, 24 aa, Artificial Sequence
  PSP94 family member
KGNKHPINSEWQTDNCETCTCYET

SEQ ID NO: 67 - protein, 25 aa, Artificial Sequence
  PSP94 family member
LKGNKHPINSEWQTDNCETCTCYET

SEQ ID NO: 68 - protein, 26 aa, Artificial Sequence
  PSP94 family member
DLKGNKHPINSEWQTDNCETCTCYET

SEQ ID NO: 69 - protein, 27 aa, Artificial Sequence
  PSP94 family member
MDLKGNKHPINSEWQTDNCETCTCYET

SEQ ID NO: 70 - protein, 28 aa, Artificial Sequence
  PSP94 family member
CMDLKGNKHPINSEWQTDNCETCTCYET

SEQ ID NO: 71 - protein, 29 aa, Artificial Sequence
  PSP94 family member
KCMDLKGNKHPINSEWQTDNCETCTCYET

SEQ ID NO: 72 - protein, 30 aa, Artificial Sequence
  PSP94 family member
RKCMDLKGNKHPINSEWQTDNCETCTCYET

SEQ ID NO: 73 - protein, 31 aa, Artificial Sequence
  PSP94 family member
TRKCMDLKGNKHPINSEWQTDNCETCTCYET

SEQ ID NO: 74 - protein, 32 aa, Artificial Sequence
  PSP94 family member
STRKCMDLKGNKHPINSEWQTDNCETCTCYET

SEQ ID NO: 75 - protein, 33 aa, Artificial Sequence
  PSP94 family member
DSTRKCMDLKGNKHPINSEWQTDNCETCTCYET

SEQ ID NO: 76 - protein, 34 aa, Artificial Sequence
  PSP94 family member
GDSTRKCMDLKGNKHPINSEWQTDNCETCTCYET

SEQ ID NO: 77 - protein, 35 aa, Artificial Sequence
  PSP94 family member
PGDSTRKCMDLKGNKHPINSEWQTDNCETCTCYET

SEQ ID NO: 78 - protein, 36 aa, Artificial Sequence
  PSP94 family member
VPGDSTRKCMDLKGNKHPINSEWQTDNCETCTCYET

SEQ ID NO: 79 - protein, 37 aa, Artificial Sequence
  PSP94 family member
GVPGDSTRKCMDLKGNKHPINSEWQTDNCETCTCYET

SEQ ID NO: 80 - protein, 38 aa, Artificial Sequence
  PSP94 family member
EGVPGDSTRKCMDLKGNKHPINSEWQTDNCETCTCYET

SEQ ID NO: 81 - protein, 39 aa, Artificial Sequence
  PSP94 family member
NEGVPGDSTRKCMDLKGNKHPINSEWQTDNCETCTCYET

SEQ ID NO: 82 - protein, 40 aa, Artificial Sequence
  PSP94 family member
PNEGVPGDSTRKCMDLKGNKHPINSEWQTDNCETCTCYET

SEQ ID NO: 83 - protein, 41 aa, Artificial Sequence
  PSP94 family member
IPNEGVPGDSTRKCMDLKGNKHPINSEWQTDNCETCTCYET

SEQ ID NO: 84 - protein, 42 aa, Artificial Sequence
  PSP94 family member
FIPNEGVPGDSTRKCMDLKGNKHPINSEWQTDNCETCTCYET

SEQ ID NO: 85 - protein, 43 aa, Artificial Sequence
  PSP94 family member
YFIPNEGVPGDSTRKCMDLKGNKHPINSEWQTDNCETCTCYET

SEQ ID NO: 86 - protein, 44 aa, Artificial Sequence
  PSP94 family member
CYFIPNEGVPGDSTRKCMDLKGNKHPINSEWQTDNCETCTCYET

SEQ ID NO: 87 - protein, 45 aa, Artificial Sequence
  PSP94 family member
SCYFIPNEGVPGDSTRKCMDLKGNKHPINSEWQTDNCETCTCYET

SEQ ID NO: 88 - protein, 15 aa, Artificial Sequence
  polypeptide analogue
XWQXDXCXXCXCXXX

SEQ ID NO: 89 - protein, 30 aa, Artificial Sequence
  PSP94 family member
EWQTDNCETCTCYETEWQTDNCETCTCYET

SEQ ID NO: 90 - protein, 45 aa, Artificial Sequence
  PSP94 family member
EWQTDNCETCTCYETEWQTDNCETCTCYETEWQTDNCETCTCYET

SEQ ID NO: 91 - protein, 60 aa, Artificial Sequence
  PSP94 family member
EWQTDNCETCTCYETEWQTDNCETCTCYETEWQTDNCETCTCYETEWQTDNCETCTCYET